(19) United States
(12) Patent Application Publication
(10) Pub. No.: US 2016/0327078 A1
(43) Pub. Date: Nov. 10, 2016

(54) OXYGEN SENSOR SEAT STRUCTURE

(71) Applicant: Mei-Pin Hsing, Changhua County (TW)

(72) Inventor: Mei-Pin Hsing, Changhua County (TW)

(21) Appl. No.: 14/705,936

(22) Filed: May 6, 2015

Related U.S. Application Data
(63) Continuation-in-part of application No. 14/704,999, filed on May 6, 2015.

(51) Int. Cl. F16B 9/02; G01M 15/02; G01N 33/00

(52) U.S. Cl. CPC F16B 9/026; G01N 33/0036; G01M 15/02

(57) ABSTRACT

An oxygen sensor seat structure used for an exhaust pipe of automobiles is disclosed. The invention includes a fixing sleeve and a tube. The fixing sleeve has a threaded hole for being installed with an oxygen sensor. The tube is connected to a first end of the fixing sleeve and is arc-shaped.

### CROSS-REFERENCE TO RELATED APPLICATIONS

This application is a continuation-in-part of U.S. patent application Ser. No. 14/704,999, filed May 6, 2015, now pending.

### BACKGROUND OF THE INVENTION

1. Technical Field

The invention relates to exhaust pipes of automobiles, particularly to junction between a catalytic converter and exhaust pipe.

2. Related Art

An oxygen sensor is installed in a rear portion of a catalytic converter for detecting functions of catalytic. The oxygen sensor will damage if the catalytic converter is removed. When the engine connecting the exhaust pipe is equipped with an engine control unit (ECU), the ECU cannot judge if the air-fuel ratio is normal or not. An engine warning light in the dashboard will light up. Even the engine may not work normally.

### SUMMARY OF THE INVENTION

An object of the invention is to provide an oxygen sensor seat structure, which can prevent an oxygen sensor from sending unnecessary data to an ECU of engine when an exhaust pipe has been refitted.

To accomplish the above objection, the oxygen sensor seat structure of the invention includes a fixing sleeve and a tube. The fixing sleeve has a threaded hole for being installed with an oxygen sensor. The tube is connected to a first end of the fixing sleeve and is arc-shaped.

### BRIEF DESCRIPTION OF THE DRAWINGS

FIG. 1 is an exploded view of the invention; and

FIG. 2 is a cross-sectional view of the invention.

### DETAILED DESCRIPTION OF THE INVENTION

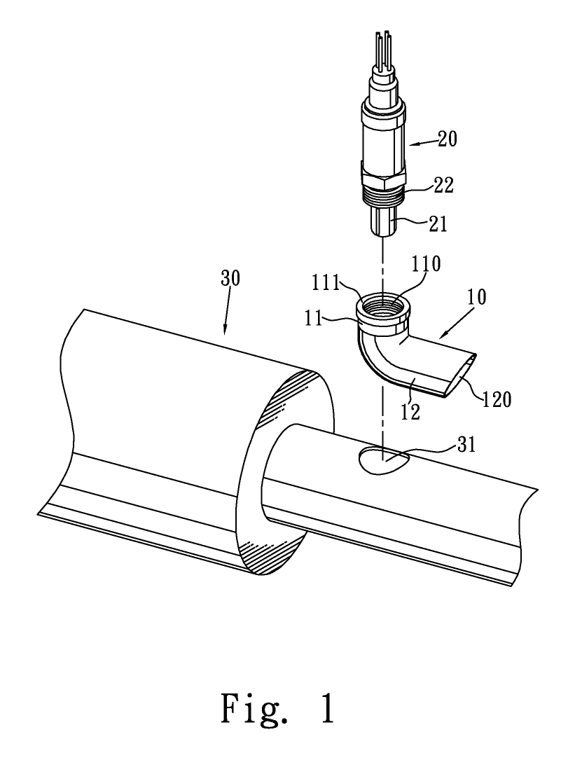

Please refer to FIG. 1. The oxygen sensor seat structure of the invention includes a fixing sleeve 11 and a tube 12. The fixing sleeve 11 has a first end and a second end. An end of the tube 12 is connected to the first end of the fixing sleeve 11. The fixing sleeve 11 has a threaded hole 110 for being screwed by a threaded end 22 of an oxygen sensor 20. The fixing sleeve 11 is fastened into a fixing hole 31 of an exhaust pipe 30. A flange 111 is formed on the second end of the fixing sleeve 11. The flange 111 is greater than the fixing hole 31 in diameter, so the flange 111 can be stopped by an edge of the fixing hole 31. The tube 12 is arc-shaped. A free end 120 of the tube 12 is inclined.

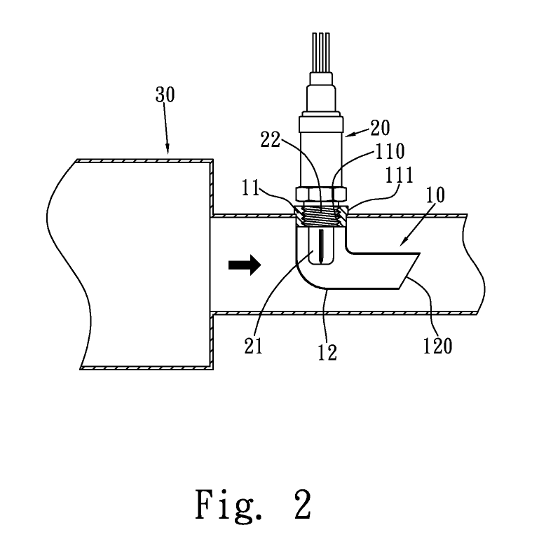

Please refer to FIG. 2. The oxygen sensor 20 has the threaded end 22 and a sensing end 21 projecting from the threaded end 22. The tube 12 is placed in the exhaust pipe 30. When the threaded end 22 is screwed in the threaded hole 110 of the fixing sleeve 11, the sensing end 21 is cloaked by the tube 12. Thus, the oxygen sensor 20 will not be influenced by the exhaust flow in the exhaust pipe 30.

It will be appreciated by persons skilled in the art that the above embodiment has been described by way of example only and not in any limitative sense, and that various alterations and modifications are possible without departure from the scope of the invention as defined by the appended claims.

What is claimed is:

1. An oxygen sensor seat structure comprising:
a fixing sleeve, having a threaded hole; and
a tube, connected to a first end of the fixing sleeve, being arc-shaped.

2. The oxygen sensor seat structure of claim 1, wherein a second end of the fixing sleeve is formed with a flange.

3. The oxygen sensor seat structure of claim 1, wherein a free end of the tube is inclined.
